(19) United States
(12) Patent Application Publication
(10) Pub. No.: US 2011/0311478 A1
(43) Pub. Date: Dec. 22, 2011

(54) Combinatorial Methods For Treating Cellular Proliferative Disorders And Immune Deficiencies Using Salicinium

(76) Inventor: Joe Ernest BROWN, Grass Valley, CA (US)

(21) Appl. No.: 13/214,022

(22) Filed: Aug. 19, 2011

Related U.S. Application Data
(63) Continuation of application No. 12/418,342, filed on Apr. 3, 2009.
(60) Provisional application No. 61/042,210, filed on Apr. 3, 2008.

(51) Int. Cl. A61K 38/20; A61K 35/32; A61K 36/28; A61K 36/87; A61K 35/20; A61K 36/185; A61K 31/706; A61K 39/395; A61K 38/50; A61K 38/19; A61K 38/18; A61K 31/122; A61K 31/7004; A61P 35/00; A61K 38/21; A61K 39/04; A61K 38/06; A61K 31/7048

(52) U.S. Cl. 424/85.2; 514/23; 424/548; 424/764; 424/766; 424/130.1; 424/535; 424/725; 514/9.2; 514/43; 424/133.1; 424/142.1; 424/94.6; 424/85.1; 514/7.7; 424/94.1; 424/85.7; 424/248.1; 514/19.3

(57) ABSTRACT

The present invention provides novel immunotherapeutic compositions and methods useful for treating or preventing microbial infections, weakened immune systems, diseases in which cells have become obligately anerobic and cellular proliferative disorders including cancer. The immunotherapeutics herein use benzaldehyde derivatives, precursors and intermediaries alone or in combination with additional therapeutic agents to stimulate the immune system and inhibit cellular proliferation. The immunotherapeutics of the present invention are particularly useful in the treatment of microbial infections and cellular proliferative disorders which are resistant to traditional methods of treatment such as antibiotics and chemotherapy

### RELATED APPLICATION

This application claims the benefit of priority from U.S. Provisional patent application Ser. No. 61/042,210, filed Apr. 3, 2008 of which the priority disclosure is incorporated herein by reference in its entirety

### BACKGROUND

Cancer remains the number two cause of mortality in the United States, resulting in over 560,000 deaths per year. (Centers for Disease Control and Prevention, FastStats 2005) Conventional treatments for cellular proliferative diseases such as cancer involve a combination of surgery, chemotherapy, hormonal therapy and/or radiation treatment to eradicate neoplastic cells in a patient. However, all of these approaches pose significant drawbacks and added risks such as increased susceptibility to infection. Additionally, despite advances in detection and treatment, many treatments such as chemotherapy make only a minor contribution to survival rates leaving mortality rates unchanged and raising into question the cost-effectiveness and impact on quality of life of such treatments. (Morgan et al., Clinical Oncology 16:549-560 (2004)). There is therefore a compelling need for the development of alternative treatments for cellular proliferative diseases including cancer.

Immunotherapy is the treatment of disease by inducing, enhancing or suppressing an immune response. There are two types of immunotherapies, active immunotherapies, which stimulate the body's own immune system to fight disease; and passive immunotherapies which use immune system components (such as antibodies) created outside of the body to fight disease.

Immunotherapy has been used in the treatment of a variety of conditions ranging from allergies to cellular proliferative diseases such as cancers. Allergen immunotherapy attempts to reduce sensitivity to allergens, i.e. suppress an immune response. Anti-microbial immunotherapy, which includes vaccination, involves activating the immune system to respond to an infectious agent. Cancer immunotherapy attempts to stimulate the immune system to reject and destroy tumors.

Immunotherapy is also used to treat microbial infections. Microbial diseases have become increasingly resistant to standard treatments such as antibiotics. Antibiotic-resistant microorganisms are increasingly associated with severe morbidity and mortality and management of life-threatening infections caused by antibiotic-resistant strains is particularly difficult, as the range of therapeutic options is very limited.

Current biological therapies and immunotherapies used to treat cellular proliferative disease such as cancer may produce side effects such as rashes or swellings, flu-like symptoms, including fever, chills and fatigue, digestive tract problems or allergic reactions. Additionally, efforts to develop cancer vaccines have met with limited success as certain tumors have developed mechanisms for suppressing the normal immune surveillance response, preventing the removal of malignant cells and decreasing the effectiveness of vaccines.

There is therefore a significant need for safe and effective methods of treating and preventing infections, cellular proliferative diseases and conditions related to cellular proliferative disorders or treatment of these disorders. Particularly, there is a need for safe and effective methods of treating infections and cellular proliferative disorders that are resistant to standard treatments, while reducing or avoiding the toxicities and/or side effects associated with conventional therapies.

### Summary of the Exemplary Embodiments of the Invention

It is therefore an object of the present invention to provide novel methods and compositions for the treatment of cellular proliferative disorders including cancer.

It is a further object of the present invention to provide novel methods and compositions for immune stimulation.

It is an additional object of the present invention to provide novel methods and compositions for immune enhancement.

It is yet another object of the present invention to provide novel methods and compositions for immunotherapy.

It is an additional object of the present invention to provide novel methods and compositions to increase the effectiveness of immune surveillance.

It is another object of the present invention to provide novel methods and compositions to inhibit anaerobic respiration in cells.

It is a further object of the present invention to provide novel methods and compositions to inhibit fermentation in cells.

It is yet another object of the present invention to provide novel methods and compositions for the reduction of pain at a tumor site.

It is a further object of the present invention to provide novel methods and compositions for strengthening the immune system.

It is yet another object of the present invention to provide novel methods and compositions for the treatment of microbial infections including bacterial, viral and fungal infections.

It is an additional object of the present invention to provide novel methods and compositions for the treatment of resistant forms of cellular proliferative disorders, including, but not limited to, stage IV or terminal cancers.

It is yet another object of the present invention to provide a means of transporting anti-cellular proliferative agents into cells using glycomes as a transporting agent.

The invention achieves these objects and satisfies additional objects and advantages by providing novel and surprisingly effective immunotherapeutic methods and compositions for use in mammalian subjects comprising benzaldehyde derivatives including, but not limited to, those represented by Formulas I-IV, intermediaries of Formulas I-IV and precursors to those benzaldehyde derivatives as represented by Formulas V-VII, below.

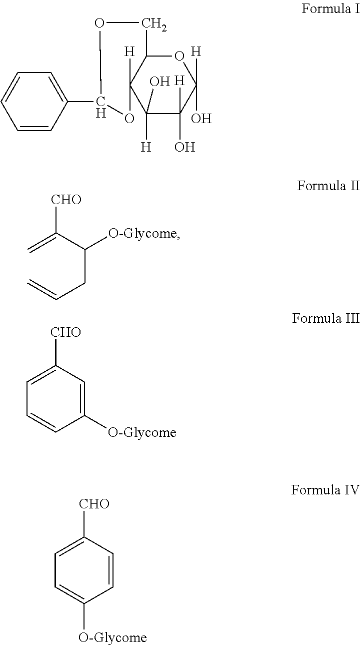

wherein the glycome or the representative glucose as shown in Formula I is a carbohydrate or sugar including, but not limited to, any one of the hexoses including, but not limited to, the α or β forms of glucose, mannose, galactose, fructose, or a biose formed from any two of the above, wherein the two hexoses may be the same or different.

Useful benzaldehyde derivatives within the formulations and methods of the invention include, but are not limited to, 4,6-O-benzylidine-D-glucopyranosyloxy, 2-β-D-glucopyaranosyloxy benzaldehyde, 3-β-D-glucopyranosyloxy benzaldehyde, and 4-β-D-glucopyranosyloxy benzaldehyde. Other useful forms of derivatives for use within the invention include other pharmaceutically acceptable active salts of said compounds, as well as active isomers, enantiomers, polymorphs, intermediaries, precursors, solvates, hydrates, and/or prodrugs of said compounds. Useful precursors and intermediaries of 4,6-O-benzylidine-D-glucopyranosyloxy, 2-β-D-glucopyaranosyloxy benzaldehyde, 3-β-D-glucopyranosyloxy benzaldehyde, and 4-β-D-glucopyranosyloxy benzaldehyde which may also be used in the methods and compositions of the present invention may include, but are not limited to, precursors such as 2(hydroxymethyl)phenyl-β-D-glucopyranoside as seen in Formula V, below, 3-(hydroxymethyl)phenyl-β-D-glucopyranoside as seen in Formula VI, below, and 4(hydroxymethyl)phyenyl-β-D-glucopyranoside as seen in Formula VII below; and intermediate compounds such as, but not limited to, 2-hydroxybenzaldehyde, 3-hydroxybenzaldehyde, and 4-hydroxybenzaldehyde which convert to salicylic acid, 3-hydroxysalicilic acid, and 4-hydroxysalicylic acid respectively, or any other pharmaceutically acceptable active salts of said compounds, as well as active isomers, enantiomers, polymorphs, intermediaries, precursors, solvates, hydrates, and/or prodrugs of said compounds.

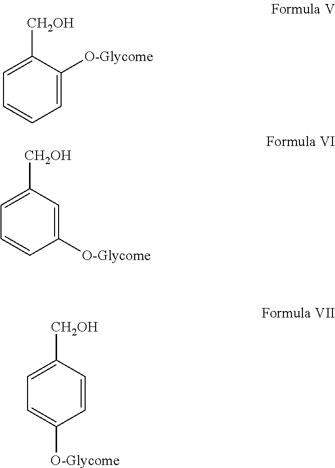

wherein the glycome may be any carbohydrate or sugar including, but not limited to any form of the hexoses, including the α and β forms of glucose, mannose, galactose, and fructose, or a biose formed from any two of the hexoses, wherein the hexoses may be the same or different.

In exemplary embodiments, the compositions and methods of the invention employ a bezaldehyde derivative compound of Formula I-IV, precursor compound of Formula V-VII, or intermediary compounds alone or in combination as immunotherapeutics. Additional embodiments may employ the compositions and methods of the invention to treat and/or prevent symptoms of cellular proliferative disorders including cancer, or other diseases and conditions associated with cancer. Further embodiments may employ the compositions and methods of the invention as antimicrobials. Still other embodiments may employ the compositions and methods of the invention as fermentation inhibiting compounds.

Mammalian subjects amenable for treatment with benzaldehyde derivatives and precursors according to the methods of the invention include, but are not limited to, subjects suffering from cellular proliferative disorders including, but not limited to, skin cancer, including, but not limited to, melanoma; breast cancer; lung cancer; thyroid cancer; esophageal cancer; sarcoma; brain cancer; prostate cancer; colorectal cancer; gastric cancer; bladder cancer; colon cancer; ovarian cancer; lymphoma; mesothelioma; pancreatic cancer; Hodgkin's disease; testicular cancer; gall bladder cancer; waldenstrom's disease; stomach cancer; pseudo mucinous peritoneii; carcinoma of the colon; cancer of the stomach; cancer of the tongue; peritonitis carcinomatosa; cancer of the liver, malignancies induced by SV₄₀ virus as well as additional cellular proliferative disorders such as psoriasis. Subjects amenable to treatment may have cellular proliferative disorders at any stage of development including, but not limited to, resistant forms of cellular proliferative diseases such as stage IV or terminal cancers or cellular proliferative disorders which otherwise do not respond or respond minimally to conventional treatments such as chemotherapy. Subjects amenable to treatment may further include human and other mammalian subjects suffering from diseases caused by cellular degradation in which the cells become obligately anaerobic. Subjects amenable to treatment may additionally include human and other mammalian subjects suffering from a weakened immune system.

Individuals suffering from cellular proliferative disorders frequently suffer from secondary infections including microbial infections such as bacterial, viral and fungal infections such as, but not limited to Lyme disease, candadiasis, Epstein Barr virus, and methicillin resistant staphylococcus infections. Combinatorial and coordinate treatment protocols of the present invention may be used to treat such secondary infections using, for example, anti-microbials which may be used in combination with a benzaldehyde derivative compound of Formula I-IV, precursor compound of Formula V-VII, or intermediary compounds.

Microbial infections may also be primary infections occurring on their own or without cellular proliferative disorders. Such microbial infections including bacterial, viral and fungal infections, include infections such as, but not limited to Lyme disease, candadiasis, Epstein Barr virus, and methicillin resistant staphylococcus infections. Combinatorial and coordinate treatment protocols of the present invention may be used to treat such s infections using, for example, anti-microbials which may be used in combination with a benzaldehyde derivative compound of Formula I-IV, precursor compound of Formula V-VII, or intermediary compounds.

These and other subjects are effectively treated, prophylactically and/or therapeutically, by administering to the subject an immunostimulating (immune surveillance promoting, fermentation inhibiting, cellular proliferative disorder treating, immune boosting, anaerobic respiration inhibiting, anti-microbial, pain relieving) effective amount of a benzaldehyde derivative of Formula I-IV, intermediary or precursor of Formula V-VII alone or in combination with other therapeutic agents such as anti-microbials or chemotherapeutic agents. The therapeutically useful methods and formulations of the invention will effectively use benzaldehyde related derivatives of Formula I-IV, intermediaries, and precursor compounds of Formulas V-VII in a variety of forms, as noted above, including any active, pharmaceutically acceptable salt of said compounds, as well as active isomers, enantiomers, polymorphs, intermediaries, precursors, solvates, hydrates, prodrugs, and/or combinations thereof 4-β-D-glucopyranosyloxy benzaldehyde is therefore employed as an illustrative embodiment of the invention within the examples herein below.

Within additional aspects of the invention, combinatorial formulations and methods are provided which employ an effective amount of a benzaldehyde derivative compound or precursor compound in combination with one or more secondary or adjunctive active agent(s) that is/are combinatorially formulated and/or coordinately administered with a benzaldehyde derivative compound to yield an immunostimulatory (immune surveillance promoting, fermentation inhibiting, cellular proliferative disorder treating, immune boosting, anaerobic respiration inhibiting, pain relieving) effective response in the subject. Exemplary combinatorial formulations and coordinate treatment methods in this context employ the benzaldehyde derivative compound in combination with one or more additional chemotherapeutics or other indicated secondary or adjunctive therapeutic agents. The secondary or adjunctive therapeutic agents used in combination with, e.g. 4-β-D-glucopyranosyloxy benzaldehyde in these embodiments may possess direct or indirect immunostimulatory (immune surveillance promoting, fermentation inhibiting, cellular proliferative disorder treating, immune boosting, anaerobic respiration inhibiting, anti-microbial, pain relieving) activity, alone or in combination with, e.g., 4-β-D-glucopyranosyloxy benzaldehyde, or may exhibit other useful adjunctive therapeutic activity in combination with, e.g., 4-β-D-glucopyranosyloxy benzaldehyde.

Useful adjunctive therapeutic agents in these combinatorial formulations and coordinate treatment methods include, for example, chemotherapeutic agents including, but not limited to, azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, Bacillus Calmette-Guérin vaccine, interleukin-2, interferon α, filgrasten, G-CSF, epoetin alfa, erythropoietin, IL-11, oprelvekin, trastuzumab, vorinostat; antibiotics; coenzyme q; palladium lipoic complexes, including, for example, poly-MVA®; antineoplastins; cartilage; hydrazine sulfate; milk thistle; electrolytes such as calcium carbonate, magnesium carbonate, sodium bicarbonate, and potassium bicarbonate; immunoglobulins; colostrum; columbianitin extracted from Lomatium Disectum; oxidizing agents including, but not limited to, cesium chloride, potassium chloride, potassium orotate and potassium aspartate, glutathione; antioxidants; reservatol; vitis vinifera L.; myricetin 3-0 galactoside; quercetin 3-0 galactocide; vitamin and mineral supplements including but not limited to, magnesium chloride, pyridoxine, vitamin B-12, B-complex vitamins, folic acid, sodium ascorbate, L-lysine, and zinc chloride; alkaline water; grapeseed extract; Arceuthobium campylopodum; and mistletoe extract. In some embodiments, a plurality of therapeutic agents may be administered, for example, a combination of a benzaldehyde derivative compound of Formula I-IV and/or intermediaries or precursor compounds of Formula VI-VII, an oxidizing agent, an immunoglobulin and a carrier medium. In one embodiment, the carrier medium is a non-corrosive base solution such as alkaline water as disclosed in U.S. Provisional Patent Application No. 60/947,633, filed Jul. 2, 2007 and U.S. patent application Ser. No. 12/167,123, filed Jul. 2, 2008 (each of which is incorporated herein by reference in its entirety). The carrier medium may function adjunctively to enhance therapeutic of prophylactic effectiveness of the formulations and methods of the invention across the range of treatment indications disclosed herein. In a further embodiment, there may be no carrier medium. Adjunctive therapies may also be used including, but not limited to, insulin potentiation therapy, radiation therapy, the Gonzalez regimen, diet, acupuncture and surgery including cryosurgery.

The forgoing objects and additional objects, features, aspects and advantages of the instant invention will become apparent from the following detailed description.

### DETAILED DESCRIPTION OF EXEMPLARY EMBODIMENTS OF THE INVENTION

The instant invention provides novel methods and compositions for stimulating and enhancing the immune system and increasing the effectiveness of immune surveillance in mammalian subjects, including individuals and in vitro, ex vivo, and in vivo mammalian cells, tissues, and organs. Such stimulation of the immune system is effective in treating myriad diseases including cellular proliferative diseases such as cancer as well as microbial infections.

A broad range of mammalian subjects, including human subjects, are amenable to treatment using the formulations and methods of the invention. These subjects include, but are not limited to, human and other mammalian subjects presenting with cellular proliferative disorders including, but not limited to, types of cancer such as skin cancer, including, but not limited to, melanoma; breast cancer; lung cancer; thyroid cancer; esophageal cancer; sarcoma; brain cancer; prostate cancer; colorectal cancer; gastric cancer; bladder cancer; colon cancer; ovarian cancer; lymphoma; mesothelioma; pancreatic cancer; Hodgkin's disease; testicular cancer; gall bladder cancer; carcinoma; sarcoma; leukemia; lymphoma; gliomas; Waldenstrom's disease; pseudo mucinous peritoneii; carcinoma of the colon; cancer of the stomach; cancer of the tongue; peritonitis carcinomatosa; cancer of the liver; malignancies induced by SV₄₀ virus as well as additional cellular proliferative disorders such as psoriasis. Subjects amenable to treatment may have cellular proliferative disorders at any stage of development including, but not limited to, resistant forms of cellular proliferative diseases such as stage IV or terminal cancers or cellular proliferative disorders which otherwise do not respond or respond minimally to conventional treatments such as chemotherapy. Subjects amenable to treatment may further include human and other mammalian subjects suffering from diseases caused by cellular degradation in which the cells become obligately anaerobic. Further subjects amenable to treatment include those with compromised or weakened immune systems whether due to disease or treatments for disease such as cancer.

Additional subjects amenable to treatment include human and other mammalian subjects suffering from microbial infections including bacterial, viral and fungal infections such as, but not limited to Lyme disease, candadiasis, Epstein Barr virus, and methicillin resistant staphylococcus infections.

The present invention additionally provides immunostimulating, (immune surveillance promoting, fermentation inhibiting, cellular proliferative disorder treating, immune boosting, anaerobic respiration inhibiting, anti-microbial, pain relieving) formulations and methods which employ derivatives of benzaldehyde or derivative compounds of Formulas I-IV, intermediate compounds or precursor compounds of Formula V-VII, above, including active pharmaceutically acceptable compounds of this description as well as various foreseen and readily provided complexes, salts, solvates, isomers, enantiomers, intermediaries, polymorphs, precursors, and prodrugs of these compounds and combinations thereof. Such formulations and methods may be used, for example, as immunostimulating compositions, for example in the prevention and treatment of cellular proliferative diseases and/or microbial infections.

Within the methods and compositions of the invention, one or more modified benzaldehyde or derivative compounds of Formula I-IV or a precursor thereof of Formula V-VII as disclosed herein is/are effectively formulated or administered as a therapeutic agent effective for treating cellular proliferative disorders and/or related disorders including cancer. In exemplary embodiments, 4-β-D-glucopyranosyloxy benzaldehyde is demonstrated for illustrative purposes to be an immunostimulatory (immune surveillance promoting, fermentation inhibiting, cellular proliferative disorder treating, immune boosting, anaerobic respiration inhibiting, anti-microbial, and pain relieving) effective agent in pharmaceutical formulations and therapeutic methods, alone or in combination with one or more adjunctive therapeutic agent(s). The present disclosure further provides additional, pharmaceutically acceptable benzaldehyde derivative compounds of Formulas I-IV, including complexes, derivatives, precursors, salts, solvates, isomers, enantiomers, intermediaries, polymorphs, and prodrugs of the compounds disclosed herein, and combinations thereof, which are effective as immunostimulatory therapeutic agents within the methods and compositions of the invention.

Within all aspects of the instant invention, additional description and related technical details pertaining to practice of the invention may be found in U.S. patent application Ser. No. 10/988,201 filed Nov. 12, 2004 which claims the benefit of U.S. Provisional Patent Application No. 60/534,702 filed Jan. 6, 2004 and U.S. Provisional Patent Application No. 60/519,657 filed Nov. 12, 2003, each of which is incorporated by reference herein in its entirety for all purposes.

Cellular proliferative disorders relate to unregulated cell division. Unlike normal cells, these cells ignore signals to stop dividing, to specialize, or to die and be shed. Defects in cells involved in cellular proliferative disorders allow them to divide, invade the surrounding tissue, and spread by way of vascular and/or lymphatic systems.

Cellular growth, including microbial growth, is fueled by metabolic processes. In humans, carbohydrate metabolism begins with digestion in the small intestine where monosaccharides are absorbed into the blood stream. In the liver and muscles, most of the glucose is changed into glycogen until needed at some later time when glucose levels are low. If energy is needed immediately, cells, including many bacteria and fungi cells, begin glycolysis.

Glycolysis is a metabolic pathway by which a 6-carbon glucose molecule is oxidized to two molecules of pyruvic acid. When oxygen is present, cells enter aerobic respiration. During aerobic respiration, ATP is produced by cells through the complete oxidation of organic compounds using oxygen. Oxygen serves as the final electron acceptor, accepting electrons that ultimately come from the energy rich organic compounds mammals consume. If oxygen is absent, many cells are still able to use glycolysis to produce ATP through fermentation and anaerobic respiration.

Under hypoxic (or partially anaerobic) conditions, for example, in overworked muscles that are starved of oxygen, pyruvate is converted to lactic acid by anaerobic respiration (also known as fermentation). In many tissues this is a last resort for energy, and most animal tissue cannot maintain anaerobic respiration for an extended length of time. During lactic acid fermentation, pyruvate and NADH are converted to lactic acid and NAD⁺. NAD⁺ is also used in glycolysis to generate ATP in which C₆H₁₂O₆+2ATP+2NAD⁺=>2 pyruvate+4ATP+2NADH.

Cancer stem cells create an acidic environment leading to the degeneration of normal, aerobic cells into fermenting cells. When normal cells are chronically deprived of oxygen, they may be unable to resume aerobic respiration and may continue anaerobic respiration or fermentation indefinitely. Fermentation is controlled by temperature, the pH reaction of the medium, the concentration of the ferment and of the substrate. No physiological controls of these qualities other than the presence of the oxidation process are known.

Benzaldehyde is a deactivator of NAD⁺. Upon delivery to the cytosol, benzaldehyde reduces NAD⁺ to NADH+H, causing NAD⁺ to close through an electrostatic strain distortion effect thereby interfering with the normal acid detoxification process and resulting in a decrease in pH due to the inability to detoxify pyruvic acid by converting it to lactic acid and removing it from the cell. The present invention uses benzaldehyde attached to a glycome or sugar to increase the presence of benzaldehyde in anaerobically respirating cells. While not wishing to be bound, it is currently believed that an increase in benzaldehyde interrupts glycolysis in fermenting cells, stopping the unregulated growth of the cells.

It is currently theorized that interference with the anaerobic respiration of tumor cells or other fermenting cells makes them more susceptible to being detected by immune surveillance and subject to a cell mediated immune response. Tumors have a number of ways of evading the typical Th-1 response. For example, tumors secrete agents, including transforming growth factor β, II-10 and prostaglandin E-2, which have been shown to promote the Th-2 immune response while suppressing the Th-1 immune response. In fact, some cancer patients exhibit enhanced expression of Th-2 cytokines or decreased expression of Th-1 cytokines in the local tumor microenvironment. The fact that malignancies have many ways of evading the Th-1 response suggests that the ability to evade this response confers a survival advantage on malignant cells. (Ichim, C V. J Transl Med. February 8; 3 (1):8. (2005)). Furthermore, a number of studies have indicated that the expression of Th-1 cytokines is associated with a favorable clinical outcome while the expression of Th-2 cytokines is associated with an unfavorable clinical outcome in cancer patients. Increasing the effectiveness of immune surveillance would lead to a decrease in tumor cells and improve the clinical outcome of patients suffering from cellular proliferative disorders.

The invention achieves these objects and satisfies additional objects and advantages by providing novel and surprisingly effective immunostimulatory methods and compositions for treating cellular proliferative disorders such as cancer, including resistant cellular proliferative disorders such as stage IV cancers in mammalian subjects using benzaldehyde derivatives including, but not limited to, those represented by Formulas I-IV below.

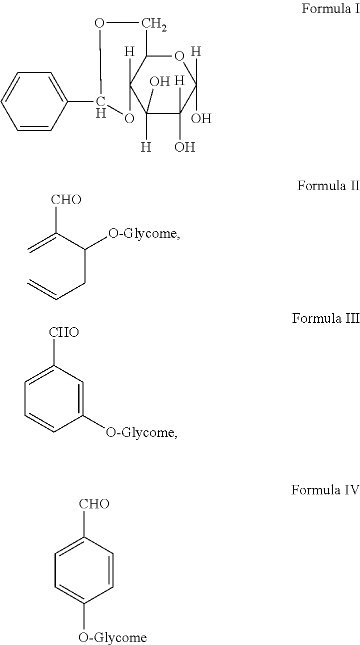

wherein the glycome or the representative glucose as shown in Formula I may be any carbohydrate or sugar including, but not limited to any one of the hexoses including, but not limited to, the α or β forms of glucose, mannose, galactose, fructose, or a biose formed from any two of the above, wherein the two hexoses may be the same or different.

Useful benzaldehyde derivatives within the formulations and methods of the invention include, but are not limited to: 4,6-O-benzylidine-D-glucopyranosyloxy, 2-β-D-glucopyaranosyloxy benzaldehyde, 3-β-D-glucopyranosyloxy benzaldehyde, and 4-β-D-glucopyranosyloxy benzaldehyde. Other useful forms of benzaldehyde derivatives for use within the invention include other pharmaceutically acceptable active salts of said compounds, as well as active isomers, enantiomers, intermediaries, polymorphs, precursors, solvates, hydrates, and/or prodrugs of said compounds.

Useful compounds may additionally include precursors of 4,6-O-benzylidine-D-glucopyranosyloxy, 2-β-D-glucopyaranosyloxy benzaldehyde, 3-β-D-glucopyranosyloxy benzaldehyde, and 4-β-D-glucopyranosyloxy benzaldehyde such as, but not limited to, 2(hydroxymethyl)phenyl-β-D-glucopyranoside as seen in Formula V, below; 3-(hydroxymethyl)phenyl-β-D-glucopyranoside as seen in Formula VI, below, and 4(hydroxymethyl)phyenyl-β-D-glucopyranoside as seen in Formula VII, below or any other pharmaceutically acceptable active salts of said compounds, as well as active isomers, enantiomers, polymorphs, intermediaries, precursors, solvates, hydrates, and/or prodrugs of said compounds.

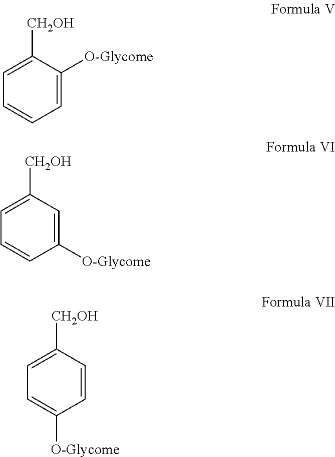

wherein the glycome may be any carbohydrate or sugar including, but not limited to any form of the hexoses, including the α and β forms of glucose, mannose, galactose, and fructose, or a biose formed from any two of the hexoses, wherein the hexoses may be the same or different.

Useful compounds may further include intermediate molecules including, but not limited to, 2-hydroxybenzaldehyde, 3-hydroxybenzaldehyde, and 4-hydroxybenzldehyde which convert to salicylic acid, 3-hydroxysaliciylic acid and 4-hydroxysalicylic acid respectively.

Immunotherapeutic compositions comprising benzaldehyde derivatives of Formulas I-IV and precursors such as those exemplified by Formulas V-VII as well as intermediary compounds, including pharmaceutical formulations of the invention, comprise an immunostimulatory amount of a benzaldehyde derivative of Formula I-IV, intermediary or precursor compound of Formula V-VII, which is effective for prophylaxis and/or treatment of cellular proliferative disorders, conditions associated with cellular proliferative disorders, and microbial disorders. Typically, an effective amount will comprise an amount of the active compound which is therapeutically effective, in a single or multiple unit dosage form, over a specified period of therapeutic intervention, to measurably alleviate one or more symptoms of cellular proliferative disorders or microbial infections in the subject, and/or to alleviate one or more symptom(s) of cellular proliferative disease, microbial infections or associated conditions in the subject. Within exemplary embodiments, these compositions are effective within in viva treatment methods to alleviate cancer.

Fermentation inhibiting compositions comprising benzaldehyde derivatives of Formulas I-IV, intermediary and precursor compounds such as those exemplified by Formulas V-VII, including pharmaceutical formulations of the invention, comprise a fermentation inhibiting effective amount of a benzaldehyde derivative compound of Formula I-IV, intermediary, or precursor compound of Formula V-VII, which is effective for prophylaxis and/or treatment of cellular proliferative disorders, microbial infection or associated conditions. Typically, an effective amount will comprise an amount of the active compound which is therapeutically effective, in a single or multiple unit dosage form, over a specified period of therapeutic intervention, to measurably alleviate one or more symptoms of cellular proliferative disorders and/or microbial infections in the subject, and/or to alleviate one or more symptom(s) of cellular proliferative disease, microbial infections, or associated condition in the subject. Within exemplary embodiments, these compositions are effective within in viva treatment methods to alleviate cancer.

Immune system strengthening compositions comprising benzaldehyde derivatives of Formulas I-IV, intermediary and precursor compounds such as those exemplified by Formulas V-VII, including pharmaceutical formulations of the invention, comprise an immune strengthening effective amount of a benzaldehyde derivative compound of Formulas I-IV and/or precursor compound of Formulas V-VII, which is effective for prophylaxis and/or treatment of weakened immune systems. Typically, an effective amount will comprise an amount of the active compound which is therapeutically effective, in a single or multiple unit dosage form, over a specified period of therapeutic intervention, to measurably alleviate one or more symptoms of immunodeficiencies in the subject, and/or to alleviate one or more symptom(s) of immunodeficiencies in the subject. Within exemplary embodiments, these compositions are effective within in vivo treatment methods to treat immunodeficiencies.

Immune surveillance promoting compositions of the invention comprising benzaldehyde derivatives of Formulas I-IV, intermediary, and precursor compounds such as those exemplified by Formulas V-VII, including pharmaceutical formulations, comprise an immune surveillance promoting effective amount of a benzaldehyde derivative compound of Formulas I-IV, intermediary and/or precursor compound of Formulas V-VII, which is effective for the promotion of immune surveillance. Typically, an effective amount will comprise an amount of the active compound which is therapeutically effective, in a single or multiple unit dosage form, over a specified period of therapeutic intervention, to measurably alleviate one or more symptoms of disease, including microbial infection and cellular proliferative disorders in the subject. Within exemplary embodiments, these compositions are effective within in vivo treatment methods to increase immune surveillance.

Pain relieving compositions of the invention comprising benzaldehyde derivatives of Formulas I-IV, intermediary, and precursor compounds such as those exemplified by Formulas V-VII, including pharmaceutical formulations, comprise a pain relieving effective amount of a benzaldehyde derivative compound of Formulas I-IV, intermediary and/or precursor compound of Formulas V-VII, which is effective for pain relief. Typically, an effective amount will comprise an amount of the active compound which is therapeutically effective, in a single or multiple unit dosage form, over a specified period of therapeutic intervention, to measurably alleviate pain. Within exemplary embodiments, these compositions are effective within in vivo treatment methods to relieve pain.

Cellular proliferative disorder treating and/or anti-microbial compositions of the invention typically comprise an effective amount or unit dosage of a benzaldehyde derivative compound of Formula I to IV, intermediary, or precursor compound of Formula V-VII, which may be formulated with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use. Cellular proliferative inhibiting effective amounts of a benzaldehyde derivative compound of Formula I-IV, intermediary and/or precursor compound (e.g., a unit dose comprising an effective concentration/amount of a compound of Formula V-VII, or of a selected pharmaceutically acceptable salt, isomer, enantiomer, intermediaries, solvate, polymorph and/or prodrug of a benzaldehyde derivative) will be readily determined by those of ordinary skill in the art, depending on clinical and patient-specific factors. Suitable effective unit dosage amounts of the active compounds for administration to mammalian subjects, including humans, may range from 10 to 10,000 mg, 1000 mg to 10,000 mg, 1000 mg to 3000 mg, 20 to 1000 mg, 25 to 750 mg, 50 to 600 mg, 150 to 550 mg, or 200 to 500 mg. In certain embodiments, the immunostimulatory (immune surveillance promoting, fermentation inhibiting, cellular proliferative disorder treating, immune boosting, anaerobic respiration inhibiting, pain relieving) effective dosage of a benzaldehyde derivative compound of Formula I-IV or precursor compound of Formula V-VII may be selected within narrower ranges of, for example, 10 to 25 mg, 30-50 mg, 75 to 100 mg, 100 to 250 mg, 250 to 500 mg, 500 to 2500 mg, 500 to 4000 mg 550 to 2000 mg, 1000 to 4000, or 2000 to 3000 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In one exemplary embodiment, dosages of 10 to 25 mg, 30-50 mg, 75 to 100 mg, 100 to 250 mg, 250 to 500 mg, 550 to 700 mg, 500 to 1000 mg, or 1000 to 3000 mg are administered one, two, three, four, or five times per day. In more detailed embodiments, dosages of 50 to 75 mg, 100 to 200 mg, 250 to 400 mg, 400 to 600 mg, 600 to 2000 mg, or 2000 to 6000 mg are administered once or twice daily. In alternate embodiments, dosages are calculated based on body weight, and may be administered, for example, in amounts from about 0.5 mg/kg to about 100 mg/kg per day, 1 mg/kg to about 75 mg/kg per day, 1 mg/kg to about 50 mg/kg per day, 2 mg/kg to about 50 mg/kg per day, 2 mg/kg to about 30 mg/kg per day, 3 mg/kg to about 30 mg/kg per day. In some embodiments, the compound may be dissolved in solution to create a solution of 0.1 to 5%, more preferably 0.9 to 3%, more preferably 1% to 2% of the benzaldehyde derivative compound of Formula I-IV and/or precursor compound of Formula V-VII.

The amount, timing and mode of delivery of compositions of the invention comprising an immunostimulatory (immune surveillance promoting, fermentation inhibiting, cellular proliferative disorder treating, immune boosting, anaerobic respiration inhibiting, anti-microbial, pain relieving) effective amount of a benzaldehyde derivative compound of Formula I-IV, intermediary, and/or precursor compound of Formula V-VII will be routinely adjusted on an individual basis, depending on such factors as weight, age, gender, and condition of the individual, the acuteness of the cellular proliferative disorder, and/or related symptoms, whether the administration is prophylactic or therapeutic, and on the basis of other factors known to effect drug delivery, absorption, pharmacokinetics, including half-life, and efficacy.

An effective dose or multi-dose treatment regimen for the instant immunostimulatory formulations will ordinarily be selected to approximate a minimal dosing regimen that is necessary and sufficient to substantially prevent or alleviate microbial infection and/or cellular proliferative diseases including cancer in the subject, and/or to substantially prevent or alleviate one or more symptoms associated with microbial infection and/or cellular proliferative disorders in the subject. A dosage and administration protocol will often include repeated dosing therapy over a course of several days or even one or more weeks or years. An effective treatment regime may also involve prophylactic dosage administered on a day or multi-dose per day basis lasting over the course of days, weeks, months or even years. Additional embodiments are described in U.S. patent application Ser. No. 10/988,201 filed Nov. 12, 2004 which claims the benefit of U.S. Provisional Patent Application No. 60/534,702 filed Jan. 6, 2004 and U.S. Provisional Patent Application No. 60/519,657 filed Nov. 12, 2003, each of which is incorporated by reference herein in its entirety for all purposes.

Various assays and model systems can be readily employed to determine the therapeutic effectiveness of immunostimulatory treatment including, but not limited to, a decrease in symptoms, a decrease in circulating endothelial cells, reduction in tumor size, collapse of the tumor, softening of the tumor, liquefaction of the tumor and a reduction in the number of circulating tumor cells.

Effectiveness of the compositions and methods of the invention may be demonstrated by a decrease in the symptoms of microbial infection. Such a decrease may be a decrease of 5%, 10%, 25%, 30%, 50%, 75%, 90% or more. Decreases may be determined by any method known to those of skill in the art, for example, through resolution of the infection, a decrease in growth of the microbe, an ELISA test, a decrease in viral count or any other method generally used to measure microbial growth/load.

Effectiveness of the compositions and methods of the invention may be demonstrated by a decrease in the symptoms of cellular proliferative disorders including a decrease in cellular proliferation, a decrease in pain, a decrease in susceptibility to infection, or any other symptom associated with cellular proliferative disorders. Such a decrease may be a decrease of 5%, 10%, 25%, 30%, 50%, 75%, 90% or more.

Effectiveness of the treatment may be monitored by counting circulating endothelial cells. Circulating endothelial cells are generally absent in the blood of healthy individuals and elevated in individuals suffering from diseases hallmarked by the presence of vascular insult such as cancer. The number of circulating endothelial cells may be determined by any means applicable such as through flow cytometry, immunobead capture, fluorescence microscopy, standard and density centrifugation, or mononuclear cell culturing on fibronectin-coated plates and immunocytochemistry. An effective amount of the compound of Formulas I-VII would decrease the number of circulating endothelial cells by 5%, 10%, 25%, 30%, 50%, 75%, 90% or more.

Effectiveness of the treatment may further be monitored by imaging such as x-rays or MRIs to determine if the size of the tumor has decreased. Effectiveness may additionally be determined by visual observation of a decrease in tumor size. In some embodiments, a decrease in tumor size may be preceded by an apparent growth of tumor size due to liquefaction of the tumor. Effective amounts of compositions containing a compound of Formula I-VII would lead to a 5%, 10%, 25%, 30%, 50%, 75%, 90% or greater reduction of tumor size. In some embodiments, effective amounts of compositions containing a compound of Formula I-VII would lead to about a 1% to about a 100% reduction in tumor size, about a 5% to about 95% reduction in tumor size, about a 10% to about a 90% reduction in tumor size, about a 15% to about an 80% reduction in tumor size; about a 15% to about a 50% reduction in tumor size. In some embodiments, effective amounts of compositions containing a compound of Formula I-VII would lead to eradication of the tumor.

Effectiveness may further be determined by measuring the number of circulating tumor cells in a sample of blood. Measurement of the number of circulating tumor cells may take place using any means applicable including, but not limited to immunomagnetic selection, flow cytometry, immunobead capture, fluorescence microscopy, cytomorphologic analysis, or cell separation technology. Levels of circulating tumor cells in a sample of blood will decrease when an effective amount of a compound of Formula I-VII is administered.

Effectiveness of treatment may further be demonstrated by a decrease in the pain associated with the cellular proliferative disorder. Pain may be measured using any of a variety of pain scales including, but not limited to, Visual analog scale, McGill Pain Questionnaire, Descriptor Differential Scale, Faces Pain Scale, Verbal Rating Scale, Simple Descriptive Pain Scale, Numerical Pain Scale (NPS), Dolorimeter Pain Index, or any other means generally used in evaluating pain.

Effectiveness of treatment may additional be demonstrated by an increase in the strength of the immune system. Such effectiveness may be demonstrated, for example by a decrease in secondary infections unrelated to the cellular proliferative disorder or microbial infection.

For each of the indicated conditions described herein, test subjects will exhibit a 10%, 20%, 30%, 50% or greater reduction, up to a 75-90%, or 95% or greater, reduction, in one or more symptom(s) caused by, or associated with, cellular proliferative disorders or conditions in the subject, compared to placebo-treated or other suitable control subjects. Within additional aspects of the invention, combinatorial cellular proliferation inhibiting and/or anti-microbial formulations and coordinate administration methods are provided which employ an effective amount of a benzaldehyde derivative of Formula I-IV or precursor compound of Formula V-VII and one or more secondary or adjunctive agent(s) that is/are combinatorially formulated or coordinately administered, or both, with the benzaldehyde derivative or precursor compound to yield a combined, multi-active anti-cellular proliferation and/or anti-microbial composition or coordinate treatment method. Exemplary combinatorial formulations and coordinate treatment methods in this context employ the benzaldehyde derivative compound or precursor compound in combination with the one or more secondary immunostimulatory, (immune surveillance promoting, fermentation inhibiting, cellular proliferative disorder treating, immune boosting, anaerobic respiration inhibiting, pain relieving) agent(s), or with one or more adjunctive therapeutic agent(s) that is/are useful for treatment or prophylaxis of the targeted (or associated) disease, condition and/or symptom(s) in the selected combinatorial formulation or coordinate treatment regimen. For most combinatorial formulations and coordinate treatment methods of the invention, a benzaldehyde derivative compound of Formula I-IV or precursor compound of Formula V-VII is formulated, or coordinately administered, in combination with one or more secondary or adjunctive therapeutic agent(s), to yield a combined formulation or coordinate treatment method that is combinatorially effective or coordinately useful as an immunostimulatory (immune surveillance promoting, fermentation inhibiting, cellular proliferative disorder treating, immune boosting, anaerobic respiration inhibiting, anti-microbial, pain relieving) agent in the subject. Exemplary combinatorial formulations and coordinate treatment methods in this context employ a benzaldehyde derivative compound of Formula I-IV, intermediary, or precursor compound of Formula V-VII in combination with one or more secondary or adjunctive therapeutic agents selected from, e.g., chemotherapeutic agents, azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, Bacillus Calmette-Guérin vaccine, interleukin-2, interferon α, filgrasten, G-CSF, epoetin alfa, erythropoietin, IL-11, oprelvekin, trastuzumab, vorinostat; antibiotics, coenzyme q; palladium lipoic complexes including, for example, poly-MVA®; antineoplastins; cartilage; hydrazine sulfate; milk thistle; electrolytes such as calcium carbonate, magnesium carbonate, sodium bicarbonate, and potassium bicarbonate; oxidizing agents, including, but not limited to, cesium chloride, potassium chloride, potassium orotate and potassium aspartate; immunoglobulins; colostrum; vitamin and mineral supplements including, but not limited to, zinc chloride, magnesium chloride, pyridoxine, vitamin B-12, B complexes, folic acid, sodium ascorbate, and L-lysine; probiotic compounds; a non-corrosive base solution or alkaline water as described in U.S. Provisional Patent Application No. 60/947,633, filed Jul. 2, 2007 and U.S. patent application Ser. No. 12/167,123, filed Jul. 2, 2008 (each of which is incorporated herein by reference in its entirety); glutathione; grapeseed extract; columbianitin extracted from Lomatium Disectum; Arceuthobium campylopodum; and mistletoe extract. Adjunctive therapies may also be used including, but not limited to, insulin potentiation therapy, radiation therapy, the Gonzalez regimen, diet, acupuncture and surgery. In some embodiments, multiple agents may be administered, for example, a combination of a benzaldehyde derivative compound of Formula I-IV or precursor compound of Formula V-VII, an oxidizing agent, an immunoglobulin and a carrier medium. In one embodiment, the carrier medium is alkaline water. In other embodiments, there may be no carrier medium. Adjunctive therapies may additionally include immunostimulatory treatments such as the use of alkaline water, a non-corrosive base for the modification of physiological pH created using calcium hydroxide as described in above-referenced U.S. Provisional Patent Application No. 60/947,633 and U.S. patent application Ser. No. 12/167,123.

In certain embodiments the invention provides combinatorial immunostimulatory (immune surveillance promoting, fermentation inhibiting, cellular proliferative disorder treating, immune boosting, anaerobic respiration inhibiting, pain relieving) formulations comprising a benzaldehyde derivative of Formula I-IV, intermediary or precursor compound of Formula V-VII and one or more adjunctive agent(s) having anti-Customer proliferative activity. Within such combinatorial formulations, the benzaldehyde derivative and the adjunctive agent(s) having anti-proliferative activity will be present in a combined formulation in anti-proliferative effective amounts, alone or in combination. In exemplary embodiments, a benzaldehyde derivative compound of Formula I-IV, intermediary or precursor compound of Formula V-VII and a non-benzaldehyde agent(s) will each be present in an immunostimulatory amount (i.e., in singular dosage which will alone elicit a detectable anti-cellular proliferative, anti-cancer, anti-malignancy, anti-fermentation response in the subject). Alternatively, the combinatorial formulation may comprise one or more of the benzaldehyde derivative compounds of Formula I-IV, intermediary and/or precursor compounds of Formula V-VII and a non-benzaldehyde agent(s) in sub-therapeutic singular dosage amount(s), wherein the combinatorial formulation comprising both agents features a combined dosage of both agents that is collectively effective in eliciting an immunostimulatory response. Thus, one or both of the benzaldehyde derivative compound of Formula I-IV, intermediary compound, or precursor compound of Formula V-VII and a non-benzaldehyde agent(s) may be present in the formulation, or administered in a coordinate administration protocol, at a sub-therapeutic dose, but collectively in the formulation or method they elicit a detectable immunostimulatory response in the subject.

To practice coordinate administration methods of the invention, a benzaldehyde derivative compound may be administered, simultaneously or sequentially, in a coordinate treatment protocol with one or more of the secondary or adjunctive therapeutic agents contemplated herein. Thus, in certain embodiments a compound is administered coordinately with a non-benzaldehyde agent, or any other secondary or adjunctive therapeutic agent contemplated herein, using separate formulations or a combinatorial formulation as described above (i.e., comprising a benzaldehyde derivative, intermediary and/or precursor compound, and a non-benzaldehyde therapeutic agent). This coordinate administration may be done simultaneously or sequentially in either order, and there may be a time period while only one or both (or all) active therapeutic agents individually and/or collectively exert their biological activities. A distinguishing aspect of all such coordinate treatment methods is that the benzaldehyde derivative compound, precursor or intermediary compound exerts at least some immunostimulatory activity, which yields a favorable clinical response in conjunction with a complementary, or distinct, clinical response provided by the secondary or adjunctive therapeutic agent. Often, the coordinate administration of the benzaldehyde derivative compound with the secondary or adjunctive therapeutic agent will yield improved therapeutic or prophylactic results in the subject beyond a therapeutic effect elicited by the benzaldehyde derivative compound, precursor or intermediary compound, or the secondary or adjunctive therapeutic agent administered alone. This qualification contemplates both direct effects, as well as indirect effects.

Within exemplary embodiments, a benzaldehyde derivative compound, precursor compound, or intermediary compound will be coordinately administered (simultaneously or sequentially, in combined or separate formulation(s)), with one or more secondary benzaldehyde agents, or other indicated therapeutic agents, e.g., selected from, for example, chemotherapeutic agents, azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, Bacillus Calmette-Guérin vaccine, interleukin-2, interferon α, filgrasten, epoetin alfa, erythropoietin, IL-11, oprelvekin, trastuzumab, and vorinostat.

Individuals undergoing treatment for cellular proliferative diseases frequently suffer from secondary infections. In some embodiments of the invention, adjunctive therapeutics such as antibiotics; coenzyme q; palladium lipoic complexes, including, for example, poly-MVA®; antineoplastins; cartilage; hydrazine sulfate; milk thistle; electrolytes such as calcium carbonate, magnesium carbonate, sodium bicarbonate, and potassium bicarbonate; antioxidants; reservatol; vitis vinifera L.; myricetin 3-0 galactoside; quercetin 3-0 galactocide; vitamin and mineral supplements including, but not limited to, magnesium chloride, pyridoxine, vitamin B-12, B-complex, folic acid, sodium ascorbate, L-lysine, and zinc chloride; glutathione; mistletoe extract; Arceuthobium campylopodum; grapeseed extract; oxidizing agents including, but not limited to, potassium chloride, potassium orotate and potassium aspartate; immunoglobulins; colostrum; columbianitin extracted from Lomatium Disectum and alkaline water, as described in above-referenced U.S. Provisional Patent Application No. 60/947,633 and 12/167,123 may be administered as part of combinatorial or coordinate treatment protocols. In some embodiments, multiple agents may be administered, for example, a combination of a benzaldehyde derivative compound of Formula I-IV, intermediary compound, or precursor compound of Formula V-VII, an oxidizing agent, an immunoglobulin and a carrier medium. In one embodiment, the carrier medium is alkaline water. Adjunctive therapies may also be used including, but not limited to, radiation therapy, insulin potentiation therapy, the Gonzalez regimen, diet, acupuncture and surgery.

In some embodiments, dosage regimes may include both combinatorial formulations and coordinate administration. Dosage regimes may include multiple units with the same or different therapeutic agents combined in each unit. For example, a benzaldehyde derivative compound of Formula I-IV, intermediary compound, or precursor compound of Formula V-VII may be combined with Arceuthobium campylopodum; and grapeseed extract in one capsule. A probiotic and immunoglobulin may be combined in a second capsule. An individual may be given one or more capsules of the benzaldehyde derivative compound combination and one or more capsules of the probiotic and immunoglobulin combination. In one embodiment, a dosage comprises two capsules of the benzaldehyde derivative compound combination and one capsule of the probiotic and immunoglobulin combination. Any combination of therapeutic agents may be administered singly or in a combination designed to achieve the desired effects.

As noted above, in all of the various embodiments of the invention contemplated herein, the malignancy treating methods and formulations may employ a benzaldehyde derivative compound in any of a variety of forms, including any one or combination of the subject compound's pharmaceutically acceptable salts, isomers, enantiomers, intermediaries, polymorphs, precursors, solvates, hydrates, and/or prodrugs. In exemplary embodiments of the invention, 4-β-D-glucopyranosyloxy benzaldehyde, is employed within the therapeutic formulations and methods for illustrative purposes.

The pharmaceutical compositions of the present invention may be administered by any means that achieve their intended therapeutic or prophylactic purpose. Suitable routes of administration for the compositions of the invention include, but are not limited to, oral, buccal, nasal, aerosol, topical, transdermal, mucosal, injectable, slow release, controlled release, iontophoresis, sonophoresis, and including all other conventional delivery routes, devices and methods. Injectable methods include, but are not limited to, intravenous, intramuscular, intraperitoneal, intraspinal, intrathecal, intracerebroventricular, intraarterial, subcutaneous and intranasal routes.

The compositions of the present invention may further include a pharmaceutically acceptable carrier appropriate for the particular mode of administration being employed. Dosage forms of the compositions of the present invention include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

If desired, the compositions of the invention can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps or other biocompatible matrices such as cholesterol.

Compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. Oral dosage forms may further include an enteric coating that is resistant to gastric juice, and which dissolves after an oral dosage form with the enteric coating passes out of the stomach and may include, for example, a polymer agent, methacrylate copolymer, cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimeth-ylammonium ethyl acrylate copolymer, and natural resins such as zein, shellac and copal collophorium. In some embodiments, the composition may be prepared as a powder.

Additional compositions of the invention can be prepared and administered in any of a variety of inhalation or nasal delivery forms known in the art. Devices capable of depositing aerosolized purified benzaldehyde derivative formulations in the sinus cavity or pulmonary alveoli of a patient include metered dose inhalers, nebulizers, sprayers, and the like. Methods and compositions suitable for pulmonary delivery of drugs for systemic effect are well known in the art. Additional possible methods of delivery include deep lung delivery by inhalation. Suitable formulations, wherein the carrier is a liquid, for administration, as for example, a nasal spray or as nasal drops, or in oral dosage forms, may include aqueous or oily solutions of benzaldehyde derivative compositions and any additional active or inactive ingredient(s).

Further compositions and methods of the invention are provided for topical administration of a benzaldehyde compound of Formula I-IV, intermediary compound, or precursor compound of Formula V-VII for the treatment of cellular proliferative disorders such as malignancy. Topical compositions may comprise benzaldehyde derivative compound of Formula I-IV, intermediary compound or precursor compound of Formula V-VII along with one or more additional active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a benzaldehyde compound of Formula I-IV, intermediary compound, or precursor compound of Formula V-VII dissolved or dispersed in a portion of a water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example, 24 hours. Transdermal delivery may also be enhanced through techniques such as sonophoresis.

Yet additional benzaldehyde derivative compositions of the invention are designed for parenteral administration, e.g. to be administered intravenously, intramuscularly, subcutaneously or intraperitoneally, including aqueous and non-aqueous sterile injectable solutions which, like many other contemplated compositions of the invention, may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. Additional compositions and formulations of the invention may include polymers for extended release following parenteral administration. The parenteral preparations may be solutions, dispersions or emulsions suitable for such administration. The subject agents may also be formulated into polymers for extended release following parenteral administration. Pharmaceutically acceptable formulations and ingredients will typically be sterile or readily sterilizable, biologically inert, and easily administered. Such polymeric materials are well known to those of ordinary skill in the pharmaceutical compounding arts. Parenteral preparations typically contain buffering agents and preservatives, and injectable fluids that are pharmaceutically and physiologically acceptable such as water, physiological saline, balanced salt solutions, aqueous dextrose, glycerol or the like. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s). In some embodiments, localized delivery of a benzaldehyde compound of Formula I-IV, intermediary compound, or precursor compound of Formula V-VII may be desired. Such localized delivery may be achieved by injecting the compound directly into the area surrounding the cellular malignancy or into the cellular malignancy itself.

In more detailed embodiments, compositions of the invention may comprise a benzaldehyde compound of Formula I-IV, intermediary compound, or precursor compound of Formula V-VII encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively; in colloidal drug delivery systems (for example, liposomes, albumin microspheres, microemulsions, nano-particles and nanocapsules); or within macroemulsions.

As noted above, in certain embodiments the methods and compositions of the invention may employ pharmaceutically acceptable salts, e.g., acid addition or base salts of the above-described benzaldehyde derivative compounds. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, tartrate, and gluconate salts. Suitable base salts are formed from bases that form non-toxic salts, for example aluminum, calcium, lithium, magnesium, potassium, sodium, zinc and diethanolamine salts.

In other detailed embodiments, the methods and compositions of the invention for employ prodrugs of benzaldehyde compound of Formula I-IV, intermediary compound, or precursor compound of Formula V-VII. Prodrugs are considered to be any covalently bonded carriers which release the active parent drug in vivo. Examples of prodrugs useful within the invention include esters or amides with hydroxyalkyl or aminoalkyl as a substituent, and these may be prepared by reacting such compounds as described above with anhydrides such as succinic anhydride.

The invention disclosed herein will also be understood to encompass methods and compositions comprising benzaldehyde compound of Formula I-IV, intermediary compound, or precursor compound of Formula V-VII using in vivo metabolic products of the said compounds (either generated in vivo after administration of the subject precursor compound, or directly administered in the form or the metabolic product itself). Such products may result for example from the oxidation, reduction, hydrolysis, amidation, esterification and the like of the administered compound, primarily due to enzymatic processes. Accordingly, the invention includes methods and compositions of the invention employing compounds produced by a process comprising contacting a benzaldehyde compound of Formula I-IV, intermediary compound, or precursor compound of Formula V-VII with a mammalian subject for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein will also be understood to encompass diagnostic compositions for diagnosing the risk level, presence, severity, or treatment indicia of, or otherwise managing a malignant disease or condition in a mammalian subject, comprising contacting a labeled (e.g., isotopically labeled, fluorescent labeled or otherwise labeled to permit detection of the labeled compound using conventional methods) benzaldehyde compound of Formula I-IV, intermediary compound, or precursor compound of Formula V-VII to a mammalian subject (e.g., to a cell, tissue, organ, or individual) at risk or presenting with one or more symptom(s) of malignancy, and thereafter detecting the presence, location, metabolism, and/or binding state (e.g., detecting binding to an unlabeled binding partner involved in benzaldehyde receptor physiology/metabolism) of the labeled compound using any of a broad array of known assays and labeling/detection methods. In exemplary embodiments, a benzaldehyde compound of Formula I-IV, intermediary compound, or precursor compound of Formula V-VII is isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. The isotopically-labeled compound is then administered to an individual or other subject and subsequently detected as described above, yielding useful diagnostic and/or therapeutic management data, according to conventional techniques.

### EXAMPLES

The benzaldehyde derivative compounds of the present invention have been demonstrated as effective in reducing tumor growth, inducing tumor shrinkage and triggering remission in mammals including humans. Case studies of treatment of humans and other animals with stage IV cancers with benzaldehyde derivatives as well as studies of the treatment of individuals with microbial infections such as Lyme Disease, Epstein Barr, Candidiasis and MRSA with columbianitin extracted from Lomatium Disectum are provided in the examples below.

### Example I

### Purification of Crude Glycome Powder

Crude extract of para-hydroxyl-benzaldehyde-O—B-D-allopyranoside was extracted from the seeds of Helicia nilagirica Beed (also known as helicid hilgirica beed). 220 g of the powder extract (crude) was then placed in 2 L beaker and 1000 ml acetone was added. The mixture was then stirred and warmed with a cold H₂O condensing coil in the top of the beaker until the mixture reached its boiling point. The mixture was then allowed to boil for 5 minutes and cooled to the point that it could be handled. The resulting warm mixture was then filtered using Whatman #1 filter paper (Middlesex, U.K.) with a 1 L receiving flask and filter. The filter cake was then washed two times with 250 ml proportions of acetone and vacuumed dry. The filter cake was then cut into cubes and placed in a warm drying oven (60°-70° C.) until the acetone evaporated.

Purity of the extract was determined by measuring the melting point of the powder. The extract was found to have a melting point of 195/199° C.

200 g of the purified powder was then placed in a 600 ml beaker, 300 ml of 99% DMSO was then added and the solution was warmed to about 70° C. Once the powder was in solution, it was filtered using a vacuum filter through a 9 cm, glass Büchner funnel (Whatman GF/B filter) into a filter flask of 500 ml. The DMSO/powder solution was then poured into a 4 L beaker containing 3200 ml distilled water at 60-70° C. and stirred. The mixture was then cooled until crystallization began and finished in a refrigerator at 2°-5° C. for about 18-24 hours. The cooled mixture is filtered through Whatman #1 paper and suctioned dry. The filter cake was then dried in a drying oven (70° C.) with a filtered air supply. The dried cake was then filtered through a U.S. series #10 stainless steel screen with an opening size of 78 thousandths of an inch.

### Example II

### Glycome Solution for Intravenous Administration

To prepare a drip solution to enhance the immune system, 4 g of the powder purified in Example I was mixed with 10 ml of 99.9% DMSO. 9 ml of the resulting solution was then injected into a solution composed of the following:

| | | |
|---|---|---|
| 0.9% Sodium Chloride USP | 0.9% | 500 ml |
| Magnesium Chloride | 1000 mg | 5 ml |
| Pyridoxine (B-6) | 200 mg | 2 ml |
| Vitamin B-12 | 2 mg | 2 ml |
| B-Complex | 1 mg | 1 ml |
| Folic Acid | 10 mg | 1 ml |
| Sodium Ascorbate | 5 gm | 10 ml |
| L-Lysine | 1000 mg | 4 ml |
| Zinc Chloride | 12.5 mg | 4 ml |
| Glutathione | 500 mg | 5 ml |

The temperature of the mixture was maintained at 102° F. to ensure proper mixing. The solution was then infused into patients over 2 hours.

### Example III

### Preparation of Solution for Injection

A 1% solution of the purified powder of Example I was dissolved in sterile water at a temperature of 102° F. The resulting solution may then be injected directly into the tumor, into the intake vein of the tumor, into a pleural cavity or the peritoneal cavity.

### Example IV

### Preparation of Dosage Capsules

600 mg gelatin capsules were prepared by combining 35 mg of Arceuthobium campylopodum (Dwarf Mistletoe), 10 mg of Vitis vinifera L., 500 mg of 4(beta-D-glucopyranosyloxy)benzldehyde and 5 mg of magnesium stearate.

### Example V

### Preparation of Additional Dosage Capsules

Colostrum and probiotics were combined in 600 mg gelatin capsules in a 50/50 mixture by volume as an additional supplement.

### Example VI

### Methods for the synthesis of 4-O-b-D-glucopyranosylbenzldehyde

5 grams of p-hydroxybenzaldehyde and 16.87 grams of tetra-O-acetyl-a-D-glucopyranosylbromide were dissolved in 41 ml quinoline (acetonitrile may also be used in greater volume.) and 5.4 grams of silver oxide was added slowly with stirring. After the exothermic reaction subsided, the stirring was maintained for 25 minutes while 27.5 ml glacial acetic acid was added. The resulting mixture was then poured into 1.2 liters of ice water. The resulting fine crystalline precipitate was then filtered with diatomaceous earth filter aid and the resulting filter cake washed with water. The washed filter cake was extracted with hot ethanol three times (250 ml each time). The ethanol extracts were concentrated in a partial vacuum which upon cooling and standing gave fine crystals, M.P. 143° C. The resulting compound (A) was deacetylated with a slight molar excess of Sodium Methoxide in anhydrous Methyl alcohol to yield 4-O-b-D-glucopyranosyl benzaldehyde

### Example VII

### Method for Making a Topical Gel from Purified Glycome

720 grams of dried Arceuthobium Campylopodum were placed in an appropriate glass or stainless steel container and 2700 ml DMSO was added. The contents were then stirred, covered, and let stand at room temperature for 24 hours. The mixture was then strained through a 40 mesh screen and the drained DMSO preserved. 2700 ml of fresh DMSO was again added to the partially extracted Arceuthobium Campylopodum. The resulting mixture was again stirred, covered and let stand for 24 hours and then drained through a 40 mesh screen, preserving the drained DMSO. 3000 ml of distilled water was then added to the partially extracted Arceuthobium campylopodum and mixed well. The mixture was then allowed to stand for 24 hours and then strained through a 40 mesh screen and the distilled water was preserved. 3000 ml of fresh distilled water was again added to the partially extracted Arceuthobium campylopodum and mixed well. The mixture was then allowed to stand for 24 hours and then strained through a 40 mesh screen taking care to preserve the distilled water.

15 grams Diatomaceous Earth was then added to the combined DMSO extracts with stirring. The resulting mixture was then vacuum filtered through a Whatman #1 filter (Middlesex, U.K.) and the filter contents preserved.

The filtered DMSO extract was placed in a 4 liter beaker and 180 g purified glycome powder was added. The mixture was then stirred until the powder was completely dissolved.

15 Grams Diatomaceous Earth was added to the distilled water preserved previously. The resulting mixture was then vacuum filtered through the same Whatman #1 filter (Middlesex, U.K) used to filter the DMSO extract. The filtered extract was then placed in a 15 liter stainless steel mixing container and 800 ml of water was added and the mixture stirred slowly. 2000 ml of a standardized mixture of 60 mg per ml of Argemone Munita which has been juiced and strained through a 40 mesh screen and was then added to the mixture. 190 to 230 g of hydroxyethyl cellulose (depending on the viscosity desired) was then added slowly and the stirring force increased as the gel thickened. A heating tape was then placed around the container and the mixture was heated to 65 C while being stirred constantly until lumps are removed. The mixture is then allowed to cool and strained to remove any remaining lumps.

### Example VIII

### Treatment Protocol

One hundred and seventy individuals with stage four cancer, (4 with AML/ALL/CML, 5 with melanoma; 1 with bladder cancer; 1 with myeloma; 4 with brain cancer; 15 with ovarian cancer; 43 with breast cancer; 5 with pancreatic cancer; 12 with colorectal cancer; 21 with prostate cancer; 4 with esophageal cancer; 4 with renal cancer; 3 with gastric cancer; 7 with sarcoma; 9 with head and neck cancer; 1 with testicular cancer; 21 with lung cancer; 2 with thyroid cancer; 6 with non Hodgkin's lymphoma/Hodgkins disease and 2 with gall bladder/ampulla) were divided into four groups. Each individual in the four groups was given para-hydroxyl-benzaldehyde-O—B-D-allopyranoside intravenously according to the formula of Example II for five days and then given six capsules prepared according to the formulation of Example IV containing a total of 3 g/day of the para-hydroxyl-benzaldehyde-O—B-D-allopyranoside and two capsules containing the formulation of Example V for two days, repeating for four weeks. They were then given six capsules formulated according to Example IV containing a total of 3 g/day of para-hydroxyl-benzaldehyde-O—B-D-allopyranoside and two capsules containing the formulation of Example V orally per day until they entered remission.

In addition to the para-hydroxyl-benzaldehyde-O—B-D-allopyranoside, groups were given no additional treatment, conventional chemotherapy, POLY-MVA® (AMARC Enterprises, Inc., Spring Valley, Calif.) (blend of Palladium and alpha-lipoic Acid, Vitamins B1, B2 and B12, Formylmethionine, Acetyl Cystiene, and trace amounts of Molybdinum, Rhodium, and Ruthenium) or a combination thereof.

One group of thirty-five was given no additional treatment. A second group of thirteen was additionally given conventional chemotherapy. A third group of fifty-five was additionally given conventional chemotherapy and Poly-MVA® (AMARC Enterprises, Inc., Spring Valley, Calif.) (blend of Palladium and alpha-lipoic Acid, Vitamins B1, B2 and B12, Formylmethionine, Acetyl Cystiene, and trace amounts of Molybdinum, Rhodium, and Ruthenium). The remaining group of sixty-seven were additionally given Poly-MVA® (AMARC Enterprises, Inc., Spring Valley, Calif.). Patients receiving the glycome solution were given the solution of Example II intravenously for five days and then given six capsules of Example IV containing a total of 3 g/day of para-hydroxyl-benzaldehyde-O—B-D-allopyranoside and two capsules containing the formulation of Example V for two days, repeating for four weeks. They were then given six capsules formulated according to Example IV containing a total of 3 g/day of para-hydroxyl-benzaldehyde-O—B-D-allopyranoside and two capsules containing the formulation of Example V orally per day until they entered remission. Of the thirty-five patients given the glycome solution of Example II alone, i.e. without conventional chemotherapeutic agents, thirteen entered complete remission, seventeen were in partial remission or stable, and five died. Of the thirteen patients receiving the para-hydroxyl-benzaldehyde-O—B-D-allopyranoside solution of Example II and low dose conventional chemotherapy, eight survived and 5 expired. Of the fifty-five patients receiving the para-hydroxyl-benzaldehyde-O—B-D-allopyranoside solution of Example II in combination with chemotherapy and Poly-MVA® (AMARC Enterprises, Inc., Spring Valley, Calif.); thirty-nine survived and sixteen died. Of the sixty-seven patients receiving the para-hydroxyl-benzaldehyde-O—B-D-allopyranoside solution of Example II in combination with Poly-MVA® (AMARC Enterprises, Inc., Spring Valley, Calif.) and no chemotherapy, forty-six survived and twenty-one died. As used herein, survival is defined as stable remission for one month. The two year survival rate for those treated with the para-hydroxyl-benzaldehyde-O—B-D-allopyranoside solution of Example II in combination with chemotherapy and Poly-MVA® (AMARC Enterprises, Inc., Spring Valley, Calif.) was 72%.

### Example IX

### Treatment of Breast Cancer

Forty-three of the patients in the study of Example VIII suffered from breast cancer. These forty-three were divided into four groups. One group of 7 was given the glycome solution of Example II; a group of five was given the glycome solution of Example II in combination with chemotherapy; a group of thirteen was given the glycome solution of Example II in combination with chemotherapy and Poly-MVA® (AMARC Enterprises, Inc., Spring Valley, Calif.) (blend of Palladium and alpha-lipoic Acid, Vitamins B1, B2 and B 12, Formylmethionine, Acetyl Cystiene, and trace amounts of Molybdinum, Rhodium, and Ruthenium); and the remaining eighteen were given a combination of the glycome solution of Example II and Poly-MVA® (AMARC Enterprises, Inc., Spring Valley, Calif.) without chemotherapy. Patients receiving the glycome solution were given a solution containing three grams of the powder isolated in Example I a day intravenously for five days then six capsules containing a total of 3 g/day of para-hydroxyl-benzaldehyde-O—B-D-allopyranoside formulated according to Example IV and two capsules containing the formulation of Example V orally for two days, repeating for four weeks. They were then given six capsules containing a total of 3 g/day of para-hydroxyl-benzaldehyde-O—B-D-allopyranoside formulated according to Example IV and two capsules containing the formulation of Example V orally per day until they entered remission.

Overall, 79% of the breast cancer patients survived for at least two years. Eighty-six percent of those treated with the glycome-benzldehyde composition survived. 100% of those treated with a combination of para-hydroxyl-benzaldehyde-O—B-D-allopyranoside and chemotherapy survived. Sixty-nine percent of those given the para-hydroxyl-benzalde hyde-O—B-D-allopyranoside composition in combination with chemotherapy and Poly-MVA® (AMARC Enterprises, Inc., Spring Valley, Calif.) survived and seventy-eight percent of those given a combination of the para-hydroxyl-benzaldehyde-O—B-D-allopyranoside and Poly-MVA® (AMARC Enterprises, Inc., Spring Valley, Calif.) survived.

### Example X

### Treatment of Dogs

Eight dogs weighing between 60 and 80 pounds with varying malignancies including neuroendocrine cancer, nasal adenocarcinoma, brain stem tumor, thymoma, and histiocytoma were treated with 6 cc of the DMSO solution of Example II diluted in 200 cc saline administered intravenously five days a week for three weeks. The dogs were then given 1 to 2 grams of methacrylate coated para-hydroxyl-benzaldehyde-O—B-D-allopyranoside in their food seven days a week until they entered remission. Six of the dogs also received acupuncture and one received acupuncture and alkaline water. All dogs saw a significant reduction or elimination of the tumor.

### Example XI

### Preparation of Alkaline Water

50,000 g of Ca(OH)₂ is added to 500 gallons of water (100 g/gal) in a polyurethane tank surrounded by strong mono-polar magnets. The mixture is stirred until maximum disassociation is achieved. The solution is then passed through a 10 micron filter to remove any particulates. 78 ml of concentrated sulfuric acid (12° Baume) per gallon, (39000 ml total) is added to a second polyurethane tank containing 500 gallons of pure water. The acid solution is circulated through ozone generators until the pH of the solution is above 7.0. The diluted sulfuric acid is added to the filtered Ca(OH)₂ solution and the reaction is allowed to go to completion. The resulting solution is passed through a 10 micron filter to remove any anhydrous calcium sulfate. The resulting mixture may then be diluted for consumption with non-chlorinated water to reach a pH of 8.5 to 12.5.

### Example XII

### Treatment of Lyme Disease

Five individuals positive for Lyme Disease were administered a solution comprising 1 liter of Alkaline water prepared as described in Example XI and diluted with non-chlorinated drinking water to a pH of 11 mixed with 200 mg of columbianitin extracted from Lomatium Disectum twice daily for three months. At the end of the three months, all five individuals tested negative for Lyme disease.

### Example XIII

### Treatment of Epstein Barr

Thirty five individuals with Epstein Barr virus were administered a solution comprising, 1 liter of Alkaline water prepared as described in Example XI and diluted with non-chlorinated drinking water to a pH of 11 mixed with 200 mg of columbianitin extracted from Lomatium Disectum, twice daily for three months. At the end of the three months, all thirty-five individuals tested negative for the Epstein Barr virus.

### Example XIV

### Treatment of Candadiasis

25 individuals with candadiasis were administered a solution comprising, 1 liter of Alkaline water prepared as described in Example XI and diluted with non-chlorinated drinking water to a pH of 11 mixed with 200 mg of columbianitin extracted from Lomatium Disectum, twice daily for three months. At the end of the three months, the skin infections had resolved though vaginal infections remained the same.

### Example XV

### Treatment of Methicillin Resistant Staphylococcus aureus (MRSA)

Three individuals suffering from MRSA were administered a solution comprising, 1 liter of Alkaline water prepared as described in Example XI and diluted with non-chlorinated drinking water to a pH of 11 mixed with 200 mg of columbianitin extracted from Lomatium Disectum, twice daily for three months. At the end of the three months, all three individuals improved considerably and one man was able to return to work.

### Example XVI

### Isolation of Immunoglobulins

Colostrum was milked from a cow during the three day period beginning the day before and ending the day after calving. A coagulating agent was mixed with the colostrum to separate the desired immunoglobulin from the fatty component of the colostrum. The liquid immunoglobulin is then passed through a 0.8 micron filter and spray dried at a temperature of less than 157° F.

### Example XVII

### Preparation of Extraction of Dwarf Mistletoe

An alcohol extraction of Dwarf Mistletoe, Arceuthobium campylopodum, was prepared to extract antioxidants myricetin-3-O-galactoside and quercitin-3-O-galactoside. The Dwarf Mistletoe was harvested and then ground into a coarse powder. The powder was then placed in an Erlenmeyer flask with 80% cold methanol. After 24 hours, the methanol was decanted and saved, and a second aqueous extraction was carried out for a further 24 hours. The combined methanol eluents were evaporated under vacuum leaving an aqueous solution.

### Example XVIII

### Insulin Potentiation Therapy

Cancer patients are given human recombinant insulin (0.3 U/kg body weight) until glucose levels are lowered to 40 to 50 mg/dL. Patients are then given 3 grams of 4,6-O-benzylidine-D-glucopyranosyloxy in 500 ml 0.9% saline. An oral glucose supplement may also be administered if needed to prevent delayed hypoglycemic symptoms

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited with the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

Groh V, Wu J, Yee C, Spies T. Tumour-derived soluble MIC ligands impair expression of NKG2D and T-cell activation. Nature; 419: 734-738 (2002).
Ichim, C V., Revisiting immunosurveillance and immunostimulation: Implications for cancer immunotherapy. J Transl Med. February 8; 3 (1):8. (2005).
Morgan G, Ward R, Barton M, The contribution of cytotoxic Chemotherapy to 5 year survival in Adult Malignancies. Clinical Oncology 16:549-560 (2004).

We claim:

1. A method for preventing or treating cellular proliferative disorders in a mammalian subject comprising administering a fermentation inhibiting effective amount of a benzaldehyde related or derivative compound of Formula I, or a pharmaceutically-acceptable salt, isomer, enantiomer, solvate, hydrate, precursor, polymorph or prodrug thereof, to said subject 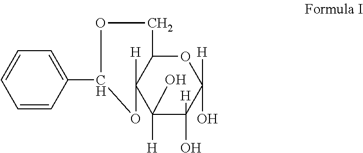
wherein the glucose is an α or β form of a hexose selected from glucose, mannose, galactose, fructose or a biose formed from two or more hexoses wherein the hexoses may be the same or different.

2. The method of claim 1, wherein Formula I is 4,6-O-benzylidine-D-glucopyranosyloxy.

3. The method of claim 1, further comprising administering a secondary anti-cellular proliferative agent or other adjunctive therapeutic agent that is effective in a combinatorial formulation or coordinate treatment regimen with said benzaldehyde derivative compound of Formula I to treat or prevent cellular proliferative disorders in said subject.

4. The method of claim 3, wherein the secondary anti-cellular proliferative disorder or adjunctive therapeutic agent is administered to said subject in a coordinate administration protocol, simultaneously with, prior to, or after administration of said benzaldehyde derivative of Formula I to said subject.

5. The method of claim 3, wherein the secondary anti-cellular proliferative disorder or adjunctive therapeutic agent is selected from the group consisting of: azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, grape seed extract, immunoglobulins, colostrum, oxidizing agents, and mistletoe.

6. The method of claim 3, wherein the secondary anti-cellular proliferative agent or adjunctive therapeutic agent is Poly-MVA®.

7. The method of claim 3, wherein the secondary anti-cellular proliferative agent or adjunctive therapeutic agent is alkaline water.

8. The method of claim 3, wherein the secondary anti-cellular proliferative agent or adjunctive therapeutic agent is electrolytes.

9. The method of claim 3, wherein the secondary anti-cellular proliferative agent or adjunctive therapeutic agent is glutathione.

10. The method of claim 1 further comprising an adjunctive therapy selected from radiation therapy, insulin potentiation therapy, the Gonzalez regimen, diet, acupuncture and surgery.

11. The method of claim 1, wherein said fermentation inhibiting effective amount comprises between about 500 to about 4000 mg of said benzaldehyde derivative compound of Formula I per day.

12. The method of claim 1, wherein said fermentation inhibiting effective amount of said benzaldehyde derivative compound of Formula I is administered one, two, three or four time per day.

13. The method of claim 1, wherein administration of said fermentation inhibiting effective amount of said benzaldehyde derivative compound of Formula I is effective to decrease tumor size in said subject by about 10% to about 90%.

14. The method of claim 1, wherein the cellular proliferative disorder is stage IV cancer.

15. The method of claim 1, wherein the cellular proliferative disorder is resistant to chemotherapeutic agents.

16. A method of controlling cellular proliferative disorders in a mammalian subject to reduce or prevent tumor growth comprising administering to said subject a fermentation inhibiting effective amount of a benzaldehyde related or derivative compound of Formula I, or a pharmaceutically-acceptable salt, isomer, precursor, enantiomer, solvate, hydrate, polymorph or prodrug thereof, to said subject 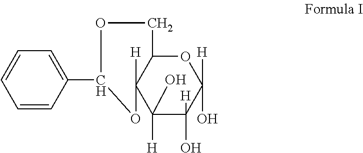
wherein the glucose is an α or β form of a hexose selected from glucose, mannose, galactose, fructose or a biose formed from two or more hexoses wherein the hexoses may be the same or different.

17. The method of claim 16, wherein the benzaldehyde derivative is 4,6-O-benzylidine-D-glucopyranosyloxy.

18. A composition for preventing or alleviating cellular proliferative disorders in a mammalian subject comprising a fermentation inhibiting effective amount of a benzaldehyde related or derivative compound of Formula I, or a pharmaceutically-acceptable salt, isomer, enantiomer, solvate, hydrate, precursor, polymorph or prodrug thereof, to said subject 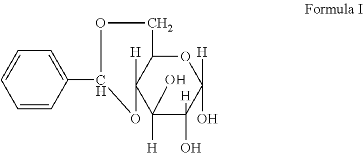
wherein the glucose is an α or β form of a hexose selected from glucose, mannose, galactose, fructose or a biose formed from two or more hexoses wherein the hexoses may be the same or different; and a secondary anti-cellular proliferative disorder agent or other adjunctive therapeutic agent useful in the treatment of cellular proliferative disorders.

19. The composition of claim 18, wherein the benzaldehyde derivative is 4,6-O-benzylidine-D-glucopyranosyloxy.

20. The composition of claim 18, wherein the secondary anti-cellular proliferative disorder or adjunctive therapeutic agent is selected from the group consisting of azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, grape seed extract, immunoglobulins, colostrum, oxidizing agents, and dwarf mistletoe.

21. The composition of claim 18, wherein the secondary anti-cellular proliferative agent or adjunctive therapeutic agent is Poly-MVA®.

22. The composition of claim 18, wherein the secondary anti-cellular proliferative agent or adjunctive therapeutic agent is alkaline water.

23. The composition of claim 18, wherein the secondary anti-cellular proliferative agent or adjunctive therapeutic agent is electrolytes.

24. The composition of claim 18, wherein the secondary anti-cellular proliferative agent or adjunctive therapeutic agent is glutathione.

25. The composition of claim 18, further comprising an adjunctive therapy selected from radiation therapy, insulin potentiation therapy, the Gonzalez regimen, diet, acupuncture and surgery.

26. The composition of claim 18, wherein said fermentation inhibiting effective amount comprises between about 500 to about 4000 mg of said benzaldehyde derivative compound of Formula I per day.

27. The composition of claim 18, wherein said fermentation inhibiting effective amount of said benzaldehyde derivative compound of Formula I is administered one, two, three or four time per day.

28. The composition of claim 18, wherein administration of said fermentation inhibiting effective amount of said benzaldehyde derivative compound of Formula I is effective to decrease tumor size in said subject by about 10% to about 90%.

29. The composition of claim 18, wherein the cellular proliferative disorder is stage IV cancer.

30. The composition of claim 18, wherein the cellular proliferative disorder is resistant to chemotherapeutic agents.

31. A method for preventing or treating cancer in a mammalian subject comprising administering a cellular-proliferative disorder inhibiting effective amount of a benzaldehyde related or derivative compound of Formula I, or a pharmaceutically-acceptable salt, isomer, enantiomer, solvate, hydrate, precursor, polymorph or prodrug thereof, to said subject 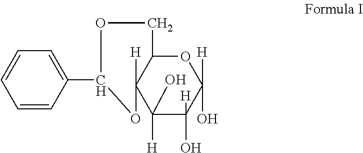
wherein the glucose is an α or β form of a hexose selected from glucose, mannose, galactose, fructose or a biose formed from two or more hexoses wherein the hexoses may be the same or different.

32. The method of claim 31, wherein Formula I is 4,6-O-benzylidine-D-glucopyranosyloxy.

33. The method of claim 31, further comprising administering a secondary anti-cellular proliferative agent or other adjunctive therapeutic agent that is effective in a combinatorial formulation or coordinate treatment regimen with said benzaldehyde derivative compound of Formula I to treat or prevent cancer in said subject.

34. The method of claim 33, wherein the secondary anti-cellular proliferative disorder or adjunctive therapeutic agent is administered to said subject in a coordinate administration protocol, simultaneously with, prior to, or after administration of said benzaldehyde derivative of Formula I to said subject.

35. The method of claim 33, wherein the secondary anti-cellular proliferative disorder or adjunctive therapeutic agent is selected from the group consisting of azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, Poly-MVA®, grape seed extract, immunoglobulins, colostrum, oxidizing agents, and Dwarf mistletoe.

36. The method of claim 31 further comprising an adjunctive therapy selected from radiation therapy, insulin potentiation therapy, the Gonzalez regimen, diet, acupuncture and surgery.

37. The method of claim 31, wherein said cellular proliferation inhibiting effective amount comprises between about 500 to about 4000 mg of said benzaldehyde derivative compound of Formula I per day.

38. The method of claim 31, wherein said cellular proliferation inhibiting effective amount of said benzaldehyde derivative compound of Formula I is administered one, two, three or four time per day.

39. The method of claim 31, wherein administration of said cellular proliferation inhibiting effective amount of said benzaldehyde derivative compound of Formula I is effective to decrease tumor size in said subject by about 10% to about 90%.

40. The method of claim 31, wherein the cancer is a stage IV cancer.

41. The method of claim 31, wherein the cancer is resistant to chemotherapyeutic agents.

42. A method of controlling cancer in a mammalian subject to reduce or prevent tumor growth comprising administering to said subject a cellular proliferation inhibiting effective amount of a benzaldehyde related or derivative compound of Formula I, or a pharmaceutically-acceptable salt, isomer, enantiomer, solvate, precursor, hydrate, polymorph or prodrug thereof, to said subject 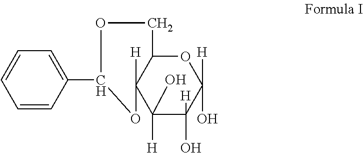
wherein the glucose is the α or β form of glucose, mannose, galactose, fructose or a biose formed from two or more hexoses wherein the hexoses may be the same or different.

43. The method of claim 42, wherein the benzaldehyde derivative is 4,6-O-benzylidine-D-glucopyranosyloxy.

44. A composition for preventing or alleviating cancer in a mammalian subject comprising a cellular proliferation inhibiting effective amount of a benzaldehyde related or derivative compound of Formula I, or a pharmaceutically-acceptable salt, isomer, enantiomer, solvate, hydrate, precursor, polymorph or prodrug thereof, to said subject 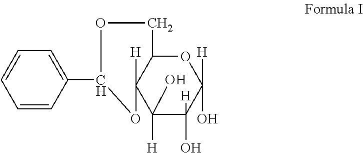
wherein the glucose is an α or β form of a hexose selected from glucose, mannose, galactose, fructose or a biose formed from two or more hexoses wherein the hexoses may be the same or different; and a secondary anti-cellular proliferative disorder agent or other adjunctive therapeutic agent useful in the treatment of cancer.

45. The composition of claim 44, wherein the benzaldehyde derivative is 4,6-O-benzylidine-D-glucopyranosyloxy.

46. The composition of claim 44, wherein the secondary anti-cellular proliferative disorder or adjunctive therapeutic agent is selected from the group consisting of: azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, oprelvekin, vorinostat, coenzyme q, palladium lipoic complexes, antineoplastins, cartilage, hydrazine sulfate, milk thistle, electrolytes, glutathione, alkaline water, Poly-MVA®, grape seed extract, immunoglobulins, colostrum, oxidizing agents, and mistletoe.

47. The composition of claim 44, wherein the secondary anti-cellular proliferative agent or adjunctive therapeutic agent is Poly-MVA.

48. The composition of claim 44, wherein the secondary anti-cellular proliferative agent or adjunctive therapeutic agent is alkaline water.

49. The composition of claim 44, wherein the secondary anti-cellular proliferative agent or adjunctive therapeutic agent is electrolytes.

50. The composition of claim 44, wherein the secondary anti-cellular proliferative agent or adjunctive therapeutic agent is glutathione.

51. The composition of claim 44, further comprising an adjunctive therapy selected from radiation therapy, insulin potentiation therapy, the Gonzalez regimen, diet, acupuncture and surgery.

52. The composition of claim 44, wherein said fermentation inhibiting effective amount comprises between about 500 mg to about 4000 mg of said benzaldehyde derivative compound of Formula I per day.

53. The composition of claim 44, wherein said fermentation inhibiting effective amount of said benzaldehyde derivative compound of Formula I is administered one, two, three or four time per day.

54. The composition of claim 44, wherein administration of said fermentation inhibiting effective amount of said benzaldehyde derivative compound of Formula I is effective to decrease tumor size in said subject by about 10% to about 90%.

55. The composition of claim 44 wherein the cancer is stage IV cancer.

56. The composition of claim 44, wherein the cancer is resistant to chemotherapeutic agents.

57. A method for preventing or treating cellular proliferative disorders in a mammalian subject comprising administering a fermentation inhibiting effective amount of a benzaldehyde related or derivative compound of Formula II, or a pharmaceutically-acceptable salt, isomer, enantiomer, solvate, hydrate, precursor polymorph or prodrug thereof, to said subject 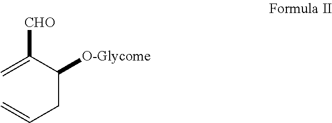
wherein the glycome is an α or β form of a hexose selected from glucose, mannose, galactose, fructose or a biose formed from two or more hexoses wherein the hexoses may be the same or different.

58. The method of claim 57, wherein Formula II is 2-β-D-glucopyranosyloxy benzaldehyde.

59. The method of claim 57, further comprising administering a secondary anti-cellular proliferative agent or other adjunctive therapeutic agent that is effective in a combinatorial formulation or coordinate treatment regimen with said benzaldehyde derivative compound of Formula II to treat or prevent cellular proliferative disorders in said subject.

60. The method of claim 59, wherein the secondary anti-cellular proliferative disorder or adjunctive therapeutic agent is administered to said subject in a coordinate administration protocol, simultaneously with, prior to, or after administration of said benzaldehyde derivative of Formula II to said subject.

61. The method of claim 59, wherein the secondary anti-cellular proliferative disorder or adjunctive therapeutic agent is selected from the group consisting of: azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, Poly-MVA®, cartilage, hydrazine sulfate, milk thistle, electrolytes, glutathione, alkaline water, Poly-MVA®, grape seed extract, immunoglobulins, colostrum, oxidizing agents, and Dwarf mistletoe.

62. The method of claim 57 further comprising an adjunctive therapy selected from radiation therapy, insulin potentation therapy, the Gonzalez regimen, diet, acupuncture and surgery.

63. The method of claim 57, wherein said fermentation inhibiting effective amount comprises between about 500 mg to about 4000 mg of said benzaldehyde derivative compound of Formula II per day.

64. The method of claim 57, wherein said fermentation inhibiting effective amount of said benzaldehyde derivative compound of Formula II is administered one, two, three or four time per day.

65. The method of claim 57, wherein administration of said fermentation inhibiting effective amount of said benzaldehyde derivative compound of Formula II is effective to decrease tumor size in said subject by about 10% to about 90%.

66. The method of claim 57, wherein the cellular proliferative disorder is stage IV cancer.

67. The method of claim 57, wherein the cellular proliferative disorder is resistant to chemotherapeutic agents.

68. A method of controlling cellular proliferative disorders in a mammalian subject to reduce or prevent tumor growth comprising administering to said subject a fermentation inhibiting effective amount of a benzaldehyde related or derivative compound of Formula II, or a pharmaceutically-acceptable salt, isomer, enantiomer, solvate, hydrate, precursor, polymorph or prodrug thereof, to said subject 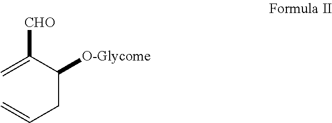
wherein the glycome is an α or β form of a hexose selected from glucose, mannose, galactose, fructose or a biose formed from two or more hexoses wherein the hexoses may be the same or different.

69. The method of claim 68, wherein the benzaldehyde derivative is 2-β-D-glucopyranosyloxy benzaldehyde.

70. A composition for preventing or alleviating cellular proliferative disorders in a mammalian subject comprising a fermentation inhibiting effective amount of a benzaldehyde related or derivative compound of Formula II, or a pharmaceutically-acceptable salt, isomer, enantiomer, solvate, hydrate, polymorph or prodrug thereof, to said subject 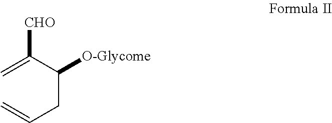
wherein the glycome is an α or β form of a hexose selected from glucose, mannose, galactose, fructose or a biose formed from two or more hexoses wherein the hexoses may be the same or different; and a secondary anti-cellular proliferative disorder agent or other adjunctive therapeutic agent useful in the treatment of cellular proliferative disorders.

71. The composition of claim 70, wherein the benzaldehyde derivative is 2-β-D-glucopyranosyloxy benzaldehyde.

72. The composition of claim 70, wherein the secondary anti-cellular proliferative disorder or adjunctive therapeutic agent is selected from the group consisting of: azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, Poly-MVA®, grape seed extract, immunoglobulins, colostrum, oxidizing agents, and mistletoe.

73. The composition of claim 70, further comprising an adjunctive therapy selected from radiation therapy, insulin potentiation therapy, the Gonzalez regimen, diet, acupuncture and surgery.

74. The composition of claim 70, wherein said fermentation inhibiting effective amount comprises between about 500 to about 4000 mg of said benzaldehyde derivative compound of Formula II per day.

75. The composition of claim 70, wherein said fermentation inhibiting effective amount of said benzaldehyde derivative compound of Formula II is administered one, two, three or four time per day.

76. The composition of claim 70, wherein administration of said fermentation inhibiting effective amount of said benzaldehyde derivative compound of Formula II is effective to decrease tumor size in said subject by about 10% to about 90%.

77. The composition of claim 70, wherein the cellular proliferative disorder is stage IV cancer.

78. The composition of claim 70, wherein the cellular proliferative disorder is resistant to chemotherapeutic agents.

79. A method for preventing or treating cellular proliferative disorders in a mammalian subject comprising administering a fermentation inhibiting effective amount of a benzaldehyde related or derivative compound of Formula III, or a pharmaceutically-acceptable salt, isomer, enantiomer, solvate, hydrate, precursor, polymorph or prodrug thereof, to said subject 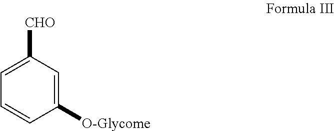
wherein the glycome is an α or β form of a hexose selected from glucose, mannose, galactose, fructose or a biose formed from two or more hexoses wherein the hexoses may be the same or different.

80. The method of claim 79, wherein Formula III is 3-β-D-glucopyranosyloxy benzaldehyde.

81. The method of claim 79, further comprising administering a secondary anti-cellular proliferative agent or other adjunctive therapeutic agent that is effective in a combinatorial formulation or coordinate treatment regimen with said benzaldehyde derivative compound of Formula III to treat or prevent cellular proliferative disorders in said subject.

82. The method of claim 81, wherein the secondary anti-cellular proliferative disorder or adjunctive therapeutic agent is administered to said subject in a coordinate administration protocol, simultaneously with, prior to, or after administration of said benzaldehyde derivative of Formula III to said subject.

83. The method of claim 81, wherein the secondary anti-cellular proliferative disorder or adjunctive therapeutic agent is selected from the group consisting of: azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, Poly-MVA®, grape seed extract, immunoglobulins, colostrum, oxidizing agents, and mistletoe.

84. The method of claim 79 further comprising an adjunctive therapy selected from radiation therapy, insulin potentiation therapy, the Gonzalez regimen, diet, acupuncture and surgery.

85. The method of claim 79, wherein said fermentation inhibiting effective amount comprises between about 500 to about 4000 mg of said benzaldehyde derivative compound of Formula III per day.

86. The method of claim 79, wherein said fermentation inhibiting effective amount of said benzaldehyde derivative compound of Formula III is administered one, two, three or four time per day.

87. The method of claim 79, wherein administration of said fermentation inhibiting effective amount of said benzaldehyde derivative compound of Formula III is effective to decrease tumor size in said subject by about 10% to about 90%.

88. The method of claim 79, wherein the cellular proliferative disorder is stage IV cancer.

89. The method of claim 79, wherein the cellular proliferative disorder is resistant to chemotherapeutic agents.

90. A method of controlling cellular proliferative disorders in a mammalian subject to reduce or prevent tumor growth comprising administering to said subject a fermentation inhibiting effective amount of a benzaldehyde related or derivative compound of Formula III, or a pharmaceutically-acceptable salt, isomer, precursor, enantiomer, solvate, hydrate, polymorph or prodrug thereof, to said subject 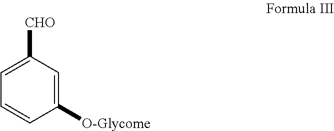
wherein the glycome is an α or β form of a hexose selected from glucose, mannose, galactose, fructose or a biose formed from two or more hexoses wherein the hexoses may be the same or different.

91. The method of claim 90, wherein the benzaldehyde derivative is 3-β-D-glucopyranosyloxy benzaldehyde.

92. A composition for preventing or alleviating cellular proliferative disorders in a mammalian subject comprising a fermentation inhibiting effective amount of a benzaldehyde related or derivative compound of Formula III, or a pharmaceutically-acceptable salt, isomer, enantiomer, solvate, hydrate, precursor, polymorph, precursor or prodrug thereof, to said subject 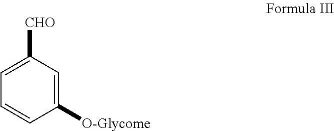
wherein the glycome is an α or β form of a hexose selected from glucose, mannose, galactose, fructose or a biose formed from two or more hexoses wherein the hexoses may be the same or different; and a secondary anti-cellular proliferative disorder agent or other adjunctive therapeutic agent useful in the treatment of cellular proliferative disorders.

93. The composition of claim 92, wherein the benzaldehyde derivative is 3-β-D-glucopyranosyloxy benzaldehyde.

94. The composition of claim 92, wherein the secondary anti-cellular proliferative disorder or adjunctive therapeutic agent is selected from the group consisting of: azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, Poly-MVA®, grape seed extract, immunoglobulins, colostrum, oxidizing agents, and Dwarf mistletoe.

95. The composition of claim 92, further comprising an adjunctive therapy selected from radiation therapy, insulin potentiation therapy, the Gonzalez regimen, diet, acupuncture and surgery.

96. The composition of claim 92, wherein said fermentation inhibiting effective amount comprises between about 500 mg to about 4000 mg of said benzaldehyde derivative compound of Formula III per day.

97. The composition of claim 92, wherein said fermentation inhibiting effective amount of said benzaldehyde derivative compound of Formula III is administered one, two, three or four time per day.

98. The composition of claim 92, wherein administration of said fermentation inhibiting effective amount of said benzaldehyde derivative compound of Formula III is effective to decrease tumor size in said subject by about 10% to about 90%.

99. The composition of claim 92, wherein the cellular proliferative disorder is stage IV cancer.

100. The composition of claim 92, wherein the cellular proliferative disorder is resistant to chemotherapeutic agents.

101. A method for preventing or treating cellular proliferative disorders in a mammalian subject comprising administering a fermentation inhibiting effective amount of a benzaldehyde related or derivative compound of Formula IV, or a pharmaceutically-acceptable salt, isomer, enantiomer, solvate, hydrate, precursor polymorph, precursor or prodrug thereof, to said subject 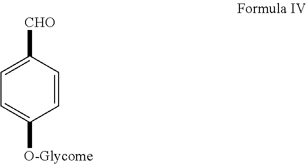
wherein the glycome is an α or β form of a hexose selected from glucose, mannose, galactose, fructose or a biose formed from two or more hexoses wherein the hexoses may be the same or different.

102. The method of claim 101, wherein Formula IV is 4-β-D-glucopyranosyloxy benzaldehyde.

103. The method of claim 101, further comprising administering a secondary anti-cellular proliferative agent or other adjunctive therapeutic agent that is effective in a combinatorial formulation or coordinate treatment regimen with said benzaldehyde derivative compound of Formula IV to treat or prevent cellular proliferative disorders in said subject.

104. The method of claim 103, wherein the secondary anti-cellular proliferative disorder or adjunctive therapeutic agent is administered to said subject in a coordinate administration protocol, simultaneously with, prior to, or after administration of said benzaldehyde derivative of Formula IV to said subject.

105. The method of claim 103, wherein the secondary anti-cellular proliferative disorder or adjunctive therapeutic agent is selected from the group consisting of: azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, Poly-MVA®, grape seed extract, immunoglobulins, colostrum, oxidizing agents, and Dwarf mistletoe.

106. The method of claim 101 further comprising an adjunctive therapy selected from radiation therapy, insulin potentiation therapy, the Gonzalez regimen, diet, acupuncture and surgery.

107. The method of claim 101, wherein said fermentation inhibiting effective amount comprises between about 500 to about 4000 mg of said benzaldehyde derivative compound of Formula IV per day.

108. The method of claim 101, wherein said fermentation inhibiting effective amount of said benzaldehyde derivative compound of Formula IV is administered one, two, three or four time per day.

109. The method of claim 101, wherein administration of said fermentation inhibiting effective amount of said benzaldehyde derivative compound of Formula IV is effective to decrease tumor size in said subject by about 10% to about 90%.

110. The method of claim 101, wherein the cellular proliferative disorder is stage IV cancer.

111. The method of claim 101, wherein the cellular proliferative disorder is resistant to chemotherapeutic agents.

112. A method of controlling cellular proliferative disorders in a mammalian subject to reduce or prevent tumor growth comprising administering to said subject a fermentation inhibiting effective amount of a benzaldehyde related or derivative compound of Formula IV, or a pharmaceutically-acceptable salt, isomer, enantiomer, solvate, hydrate, polymorph, precursor or prodrug thereof, to said subject 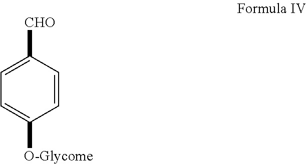
wherein the glycome is an α or β form of a hexose selected from glucose, mannose, galactose, fructose or a biose formed from two or more hexoses wherein the hexoses may be the same or different.

113. The method of claim 112, wherein the benzaldehyde derivative is 4-β-D-glucopyranosyloxy benzaldehyde.

114. A composition for preventing or alleviating cellular proliferative disorders in a mammalian subject comprising a fermentation inhibiting effective amount of a benzaldehyde related or derivative compound of Formula IV, or a pharmaceutically-acceptable salt, isomer, enantiomer, solvate, hydrate, precursor, polymorph or prodrug thereof, to said subject 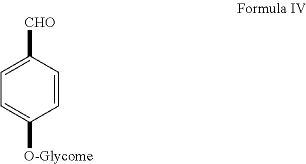
wherein the glycome is an α or β form of a hexose selected from glucose, mannose, galactose, fructose or a biose formed from two or more hexoses wherein the hexoses may be the same or different; and a secondary anti-cellular proliferative disorder agent or other adjunctive therapeutic agent useful in the treatment of cellular proliferative disorders.

115. The composition of claim 114, wherein the benzaldehyde derivative is 4-β-D-glucopyranosyloxy benzaldehyde.

116. The composition of claim 114, wherein the secondary anti-cellular proliferative disorder or adjunctive therapeutic agent is selected from the group consisting of: azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, paclitaxel albumin-stabilized nanoparticle formulation, palifermin, panitumumab, pegaspargase, pemetrexed disodium, rituximab, sorafenib tosylate, sunitinib malate, tamoxifen citrate, targretin, temozolomide, thalidomide, topotecan hydrochloride, trastuzumab, Bacillus Calmette-Guérin vaccine, interleukin-2, interferon α, rituximab, trastuzumab, filgrasten, G-CSF, epoetin alfa, erythropoietin, IL-11, oprelvekin, vorinostat, coenzyme q, palladium lipoic complexes, antineoplastins, cartilage, hydrazine sulfate, milk thistle, electrolytes, glutathione, alkaline water, Poly-MVA®, grape seed extract, immunoglobulins, colostrum, oxidizing agents, and Dwarf mistletoe.

117. The composition of claim 114, further comprising an adjunctive therapy selected from radiation therapy, insulin potentiation therapy, the Gonzalez regimen, diet, acupuncture and surgery.

118. The composition of claim 114, wherein said fermentation inhibiting effective amount comprises between about 500 to about 4000 mg of said benzaldehyde derivative compound of Formula IV per day.

119. The composition of claim 114, wherein said fermentation inhibiting effective amount of said benzaldehyde derivative compound of Formula IV is administered one, two, three or four time per day.

120. The composition of claim 114, wherein administration of said fermentation inhibiting effective amount of said benzaldehyde derivative compound of Formula IV is effective to decrease tumor size in said subject by about 10% to about 90%.

121. The composition of claim 114, wherein the cellular proliferative disorder is stage IV cancer.

122. The composition of claim 114, wherein the cellular proliferative disorder is resistant to chemotherapeutic agents.
